(19) United States
(12) Patent Application Publication
(10) Pub. No.: US 2008/0107725 A1
(43) Pub. Date: May 8, 2008

(54) Pharmaceutical Solid Dosage Forms Comprising Amorphous Compounds Micro-Embedded in Ionic Water-Insoluble Polymers

(76) Inventors: Antonio Aquino Albano, Clifton, NJ (US); Wantanee Phuapradit, Montville, NJ (US); Navnit Hargovindas Shah, Clifton, NJ (US); Zhongshui Yu, Bridgewater, NJ (US); Lin Zhang, Nutley, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/866,102

(22) Filed: Oct. 2, 2007

Related U.S. Application Data
(60) Provisional application No. 60/851,852, filed on Oct. 13, 2006.
(60) Provisional application No. 60/954,401, filed on Aug. 7, 2007.

(51) Int. Cl. A61K 9/48; A61K 31/4965

(52) U.S. Cl. 424/451; 514/255.06

(57) ABSTRACT

The present invention provides novel pharmaceutical solid dosage forms for oral administration comprising a therapeutically effective amount of an unstable crystalline form or an amorphous form of a therapeutically effective compound micro-embedded into an ionic water-insoluble polymer. The therapeutically effective compounds, which have a tendency to gel, are micro-embedded into an ionic water-insoluble polymer matrix to provide a dosage form having rapid, reproducible, and complete dissolution profiles. These novel solid pharmaceutical dosage forms are useful in the treatment or control of a number of diseases. The present invention also provides a method for treating a disease comprising administering to a subject, in need thereof, a therapeutically effective amount of the novel solid pharmaceutical dosage form. The present invention further provides a method for preparing the pharmaceutical dosage forms.

### PRIORITY TO RELATED APPLICATION(S)

This application claims the benefit of U.S. Provisional Application No. 60/851,852, filed Oct. 13, 2006, and U.S. Provisional Application No. 60/954,401 filed Aug. 7, 2007. The entire contents of the above-identified applications are hereby incorporated by reference.

### FIELD OF THE INVENTION

The present invention provides novel pharmaceutical solid dosage forms for oral administration comprising a therapeutically effective amount of an unstable crystalline form or an amorphous form of a therapeutically effective compound micro-embedded into an ionic water-insoluble polymer. The therapeutically effective compounds, which have a tendency to gel, are micro-embedded into an ionic water-insoluble polymer matrix to provide a dosage form having rapid, reproducible, and complete dissolution profiles. These novel solid pharmaceutical dosage forms are useful in the treatment or control of a number of diseases. The present invention also provides a method for treating a disease comprising administering to a subject, in need thereof, a therapeutically effective amount of the novel solid pharmaceutical dosage form. The present invention further provides a method for preparing the pharmaceutical dosage forms.

All documents cited herein are hereby expressly incorporated herein by reference.

### BACKGROUND OF THE INVENTION

Many therapeutically active compounds exist in amorphous forms, which lack the long-range order of molecular packing generally exhibited by crystalline forms. Therapeutically active amorphous compounds typically exhibit higher solubility and higher dissolution rates and thereby provide higher bioavailability than crystalline compounds. However, amorphous compounds present many difficulties associated with their instability and processability. Amorphous compounds tend to be more sensitive to manufacturing processing conditions such as high temperature and moisture levels, shearing, and increased drug loading. Amorphous compounds often gel during the manufacturing process making it very difficult to manufacture amorphous compound in the solid dosage form with reproducible dissolution rates. Many unstable crystalline forms of therapeutically effective compounds also have a tendency to gel during the manufacturing process and present similar physical stability and dissolution problems. Amorphous compounds also often require special packaging because of their relatively high hygroscopicity.

Since therapeutically active compounds in a solid unit dosage form are preferred for oral administration, it would be useful to provide methods for overcoming the gelling issues of amorphous compounds and unstable crystalline forms of therapeutically effective compounds during the manufacturing process to maintain desirable dissolution properties.

### SUMMARY OF THE INVENTION

The present invention provides a pharmaceutical solid dosage form for oral administration comprising a therapeutically effective amount of an unstable crystalline form or an amorphous form of a therapeutically effective compound micro-embedded into an ionic water-insoluble polymer, wherein the ratio of the therapeutically effective compound to the ionic water-insoluble polymer is from about 5:1 to about 1:5, respectively.

The present invention also provides a method for treating a disease comprising administering to a subject, in need thereof, a solid pharmaceutical dosage form for oral administration comprising a therapeutically effective amount of an unstable crystalline form or an amorphous form of a therapeutically effective compound micro-embedded into an ionic water-insoluble polymer, wherein the ratio of the therapeutically effective compound to the ionic water-insoluble polymer is from about 5:1 to about 1:5, respectively.

The present invention further provides a method for preparing a pharmaceutical solid dosage form for oral administration which comprises micro-embedding a therapeutically effective amount of an unstable crystalline form or an amorphous form of a therapeutically effective compound into an ionic water-insoluble polymer, wherein the ratio of the amorphous compound to the ionic polymer carrier is from about 5:1 to about 1:5, respectively.

### BRIEF DESCRIPTION OF DRAWINGS

FIG. 1 is a diagram illustrating a preferred micro-embedding process for depositing an ethanolic solution of a therapeutically effective compound and an ionic water-insoluble polymer on a microcrystalline cellulose sphere using a fluid bed coater.

FIG. 2 is a graph illustrating the powder X-Ray pattern of the pharmaceutical solid dosage form of amorphous 2(R)-(3-chloro-4-methanesulfonyl-phenyl)-3-[1(R)-3-oxo-cyclopentyl]-N-(pyrazin-2-yl)-propionamide (Compound A) (Example 3) compared to the isopropanol solvate (Compound A IPA), a physically unstable crystalline form used as a starting material, indicating that the selected micro-embedding process preferentially converted the crystalline form to amorphous form.

FIG. 3 is a graph illustrating the powder X-Ray patterns of the pharmaceutical solid dosage form of amorphous 2(R)-(3-chloro-4-methanesulfonyl-phenyl)-3-cyclopentyl-N-[5-(1(S),2-dihydroxyethyl)-pyrazin-2-yl]-propionamide (Compound B) (Example 8) compared to the physically unstable crystalline form of Compound B used as a starting material, indicating that the selected micro-embedding process preferentially converted the crystalline form to amorphous form.

FIG. 4 is a graph illustrating the dissolution profiles of the inventive pharmaceutical solid dosage form of amorphous 2(R)-(3-chloro-4-methanesulfonyl-phenyl)-3-[1(R)-3-oxo-cyclopentyl]-N-(pyrazin-2-yl)-propionamide (Compound A) micro-embedded into an ionic water-insoluble polymer (Example 1) compared to a conventional amorphous solid dosage form using a nonionic water-soluble polymer (Example 2).

FIG. 5 is a graph illustrating the dissolution profiles of the pharmaceutical solid dosage form of amorphous 2(R)-(3-chloro-4-methanesulfonyl-phenyl)-3-[1(R)-3-oxo-cyclopentyl]-N-(pyrazin-2-yl)-propionamide (Compound A) micro-embedded into an ionic water-insoluble polymers (Examples 4-5) compared to a conventional amorphous solid dosage form using nonionic water-soluble polymers (Examples 6-7).

FIG. 6 is a graph illustrating the dissolution profiles of the pharmaceutical solid dosage form of amorphous 2(R)-(3-chloro-4-methanesulfonyl-phenyl)-3-cyclopentyl-N-[5-(1(S),2-dihydroxyethyl)-pyrazin-2-yl]-propionamide (Compound B) micro-embedded into an ionic water-insoluble polymer (Example 8) compared to a conventional amorphous solid dosage form using a nonionic water-soluble polymer (Example 9).

FIG. 7 is a graph illustrating the dissolution profiles of a pharmaceutical solid dosage form of amorphous 2(R)-(3-chloro-4-methanesulfonyl-phenyl)-3-[1(R)-3-oxo-cyclopentyl]-N-(pyrazin-2-yl)-propionamide (Compound A) (Example 3) during storage, indicating no changes in dissolution profiles.

FIG. 8 is a graph illustrating the dissolution profiles of the pharmaceutical solid dosage form of amorphous 2(R)-(3-chloro-4-methanesulfonyl-phenyl)-3-cyclopentyl-N-[5-(1(S),2-dihydroxyethyl)-pyrazin-2-yl]-propionamide (Compound B) (Example 8) during storage, indicating no changes in dissolution profiles.

FIG. 9 is a graph illustrating the powder X-Ray patterns of the pharmaceutical solid dosage form of amorphous 2(R)-(3-chloro-4-methanesulfonyl-phenyl)-3-[1(R)-3-oxo-cyclopentyl]-N-(pyrazin-2-yl)-propionamide (Compound A) (Example 3) after 3-months storage at accelerated conditions (40° C./75% RH) in an induction-sealed opaque high density polyethylene bottle with a plastic cap, indicating that the compound remained in an amorphous form.

FIG. 10 is a graph illustrating the powder X-Ray patterns of a pharmaceutical solid dosage form of amorphous 2(R)-(3-chloro-4-methanesulfonyl-phenyl)-3-cyclopentyl-N-[5-(1(S),2-dihydroxyethyl)-pyrazin-2-yl]-propionamide (Compound B) (Example 8) after 6-month storage at accelerated conditions (40° C./75% RH) in an induction-sealed opaque high density polyethylene bottle with a plastic cap, indicating that the compound remained in an amorphous form.

FIG. 11 is a graph illustrating a comparison between the dissolution profiles of the inventive pharmaceutical solid dosage form of Compound A prepared by the micro-embedding process in Examples 4-5 and the solid dosage form of Compound A prepared in Examples 10-11 by a conventional process.

FIG. 12 is a graph illustrating a comparison between the dissolution profiles of the inventive pharmaceutical solid dosage form of Compound B prepared by the micro-embedding process in Example 8 and the solid dosage form of Compound B prepared in Example 12 by a conventional process.

### DETAILED DESCRIPTION OF INVENTION

The present invention provides pharmaceutical solid dosage forms for oral administration comprising a therapeutically effective amount of an unstable crystalline form or an amorphous form of a therapeutically effective compound micro-embedded into an ionic water-insoluble polymer. The therapeutically active compounds, which have a tendency to gel when exposed to aqueous media, heat and shear, cannot generally be processed by means of conventional aqueous wet granulation processes to achieve a rapid, reproducible and complete drug release. The therapeutically effective compounds of the present invention, which have a tendency to gel, are converted into an amorphous form by micro-embedding the compounds into an ionic water-insoluble polymer matrix, which provides a dosage form having rapid, reproducible, and complete dissolution profiles. The amorphous form is micro-embedded into the ionic water-insoluble polymer matrix to protect it from the manufacturing process and the environment. The novel pharmaceutical solid dosage forms may be manufactured reproducibly and are released in a uniform dissolution profile maximizing bioavailability and minimizing variability. The novel pharmaceutical solid dosage forms are preferably prepared in capsule dosage form to provide a relatively faster and more reproducible dissolution profile.

As used herein, the following terms have the given meanings:

The term “amorphous form” refers to compounds that lack the long-range order of molecular packing and have a tendency to gel when exposed to aqueous media because of their inherent physical properties, such as having a tendency to be plasticized by water.

The term “ionic polymer” refers to large molecules having a molecular weight of about 10,000, or greater, composed of many smaller molecules (monomers) covalently bonded together. These ionic polymers are practically insoluble in water but may become ionized and soluble either above or below certain pH values.

The term “ionic polymer matrix” refers to a mass of ionic polymers consisting of a number of chains, which often become entangled. A “matrix” is also defined as something within which something else originates or develops.

The term “micro-embedded” refers to a process that converts an unstable crystalline form or an amorphous form of a therapeutically active compound into amorphous form and encloses the compound closely, as if in a matrix, into the ionic water-insoluble polymer to protect the compound from the manufacturing process and the environment.

The term “pharmaceutically acceptable,” such as pharmaceutically acceptable carriers, excipients, etc., means pharmacologically acceptable and substantially non-toxic to the subject to which the particular compound is administered.

The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium, and quaternary ammonium hydroxides, such as for example, tetramethylammonium hydroxide. Chemical modification of a pharmaceutical compound (i.e., drug) into a salt is a technique well known to pharmaceutical chemists to obtain improved physical and chemical stability, hygroscopicity, and solubility of compounds. See, e.g., H. Ansel et. al., Pharmaceutical Dosage Forms and Drug Delivery Systems (6^{th} Ed. 1995) at pp. 196 and 1456-1457.

The term “prodrug” refers to compounds, which undergo biotransformation prior to exhibiting their pharmacological effects. The chemical modification of drugs to overcome pharmaceutical problems has also been termed “drug latentiation.” Drug latentiation is the chemical modification of a biologically active compound to form a new compound, which upon in vivo enzymatic attack will liberate the parent compound. The chemical alterations of the parent compound are such that the change in physicochemical properties will affect the absorption, distribution and enzymatic metabolism. The definition of drug latentiation has also been extended to include nonenzymatic regeneration of the parent compound. Regeneration takes place as a consequence of hydrolytic, dissociative, and other reactions not necessarily enzyme mediated. The terms prodrugs, latentiated drugs, and bio-reversible derivatives are used interchangeably. By inference, latentiation implies a time lag element or time component involved in regenerating the bioactive parent molecule in vivo. The term prodrug is general in that it includes latentiated drug derivatives as well as those substances, which are converted after administration to the actual substance, which combines with receptors. The term prodrug is a generic term for agents, which undergo biotransformation prior to exhibiting their pharmacological actions.

The term “therapeutically effective amount” means an amount of a therapeutically effective compound, or a pharmaceutically acceptable salt thereof, which is effective to treat, prevent, alleviate or ameliorate symptoms of a disease.

The term “therapeutically effective compound” refers to compounds that are effective to treat, prevent, alleviate or ameliorate symptoms of a disease. The therapeutically effective compounds in the present invention exist in either amorphous form or a physically unstable crystalline form and have a tendency to gel.

The term “physically unstable crystalline form” refers to crystal forms of the therapeutically active compounds that: (i) have a tendency to gel when exposed to water and/or heat; and (ii) are readily converted into an amorphous form. Physically unstable crystalline forms and amorphous forms can be distinguished by X-ray diffraction analysis.

The present invention provides pharmaceutical solid dosage forms for oral administration comprising a therapeutically effective amount of an unstable crystalline form or an amorphous form of a therapeutically effective compound micro-embedded into an ionic water-insoluble polymer. Preferably, the pharmaceutical dosage form is administered to a mammal; more preferably, the pharmaceutical dosage form is administered to a human.

The unstable crystalline forms or amorphous forms of the therapeutically effective compounds in the present invention may be selected from a wide variety of compounds and the pharmaceutically acceptable salts thereof. The amorphous compounds lack the long-range order of molecular packing and having a tendency to gel when exposed to aqueous media. The unstable crystalline compounds are physically unstable and also have a tendency to gel. Preferred therapeutically effective compounds are glucokinase activator compounds, which are compounds developed for the primary indication treatment of type 2 diabetes mellitus and future indications impairing fasting glucose (IFG) and impaired glucose tolerance (IGT). Preferred glucokinase activator compounds are 2(R)-(3-chloro-4-methanesulfonyl-phenyl)-3-[1(R)-3-oxo-cyclopentyl]-N-(pyrazin-2-yl)-propionamide (Compound A) and 2(R)-(3-chloro-4-methanesulfonyl-phenyl)-3-cyclopentyl-N-[5-(1(S),2-dihydroxyethyl)-pyrazin-2-yl]-propionamide (Compound B).

One preferred glucokinase activator compounds is 2(R)-(3-chloro-4-methanesulfonyl-phenyl)-3-[1(R)-3-oxo-cyclopentyl]-N-(pyrazin-2-yl)-propionamide (Compound A):

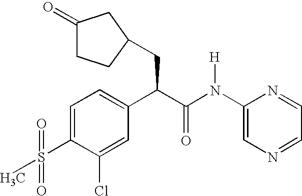

The preparation of Compound A (amorphous) is disclosed in U.S. Pat. No. 7,105,671, which disclosure is incorporated by reference herein. The preparation of Compound A IPA (isopropanol solvate) is disclosed in U.S. provisional patent application No. 60/791,256, which disclosure is incorporated by reference herein.

Another preferred glucokinase activator compounds is 2(R)-(3-chloro-4-methanesulfonyl-phenyl)-3-cyclopentyl-N-[5-(1(S),2-dihydroxyethyl)-pyrazin-2-yl]-propionamide (Compound B):

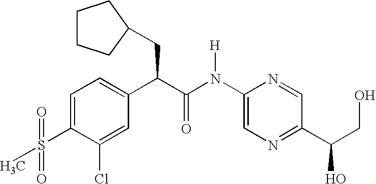

The preparation of Compound B is disclosed in United States published patent application no. 2004/0147748, which disclosure is incorporated by reference herein.

The ionic water-insoluble polymers in the present invention may be selected from a wide variety of compounds. The ionic water-insoluble polymer may be anionic or cationic. Selection of the ionic water-insoluble polymer is critical to micro-embedded the unstable crystalline form or amorphous form of the therapeutically effective compound into a matrix to prevent the compound from gelling when exposed to manufacturing condition or dissolution medium. Suitable ionic water-insoluble polymers are those generally having a molecular weight ranging from 60,000-300,000 Daltons (D), preferably 65,000-275,000 D, and most preferably 70-250,000 D. Nonlimiting illustrative examples of useful ionic water-insoluble polymers include methacrylic acid and ethyl acrylate copolymers (Eudragit® L100-55), methacrylic acid and methylmethacrylate copolymers (Eudragit® L100, Eudragit® S-100), dimethylaminoethylmethacrylate and neutral methacrylic ester copolymers (Eudragit® E100), cellulose acetate phthalates, polyvinyl acetate phthalates, hydroxylpropyl methyl cellulose phthalates, and hydroxylpropyl methyl cellulose acetate succinates.

Eudragit® L100-55 is soluble at a pH above 5.5 and is practically insoluble at a pH below 5.5. The molecular weight of Eudragit® L100-55 is approximately 250,000 D and the glass transition temperature is about 110° C. The molecular weight of Eudragit® L100 is approximately 135,000 D and the glass transition temperature is about 150° C. Eudragit® S 100 is soluble at a pH above 5 and is practically insoluble at a pH below 4.5. The molecular weight of Eudragit® S 100 is approximately 135,000 D and the glass transition temperature is about 160° C. Eudragit® E100 is a copolymer of dimethylaminoethylmethacrylate and neutral methacrylic esters. Eudragit® E100 is soluble at a pH up to 4 and is practically insoluble at a pH above 4. The molecular weight of Eudragit® E100 is approximately 150,000 D and the glass transition temperature is about 50° C. Eudragit® polymers are available from Degussa, a polymer division of Rohm & Hass GmbH.

The micro-embedding method for converting an unstable crystalline form or an amorphous form of a therapeutically effective compound into the ionic water-insoluble polymeric matrix to protect the compound from the environment may be carried out by a number of methods. Illustrative non-limiting micro-embedding methods include fluid bed coating, spray drying, lyophilizing, solvent-controlled microprecipitation, hot melt extrusion, and supercritical fluid evaporation.

In a spray drying or lyophilizing method, therapeutically effective compound, in either a physically unstable crystalline form or an amorphous form, and the ionic water-insoluble polymer are dissolved in a common solvent having a low boiling point, e.g., ethanol, acetone, etc. The solution is then spray dried or lyophilized to evaporate the solvent leaving the therapeutically effective compound micro-embedded in an amorphous form in the ionic water-insoluble polymer.

In a solvent controlled microprecipitation method, the therapeutically effective compound, in either a physically unstable crystalline form or an amorphous form, and the ionic water-insoluble polymer are dissolved in a common solvent, e.g., dimethylacetamide, dimethylformamide, ethanol, acetone, etc. The therapeutically effective compound and ionic water-insoluble polymer solution is then added to cold water (2°-5° C.) adjusted to an appropriate pH to cause the therapeutically effective compound to microprecipitate in the polymeric matrix. The desired pH of the solution is dependent upon the polymer employed and is readily ascertainable to one skilled in the art. The microprecipitate is then washed several times with the aqueous medium until the amount of residual solvent in the polymer is reduced to an acceptable limit for that solvent. An “acceptable limit” for each solvent is determined pursuant to the International Conference on Harmonization (ICH) guidelines.

In a hot melt extrusion process, the therapeutically effective compound, in either a physically unstable crystalline form or an amorphous form, and the ionic water-insoluble polymer are mixed in a blender and fed continuously to a temperature-controlled extruder causing the therapeutically effective compound to be molecularly dispersed in the molten ionic water-insoluble polymer. The resulting extrudate is cooled to room temperature and milled into a fine powder. Plasticizers may be added to lower the glass transition temperature of the polymer reducing the processing temperature.

In supercritical fluid evaporation, the therapeutically effective compound, in either a physically unstable crystalline form or an amorphous form, and the ionic water-insoluble polymer are dissolved in a supercritical fluid such as liquid nitrogen or liquid carbon dioxide. The supercritical fluid is then removed by evaporation leaving the therapeutically effective compound microprecipitated in amorphous form in the polymeric matrix.

Fluid bed coating is the most preferred micro-embedding method to provide intimate contact between an amorphous compound and an ionic water-insoluble polymer. Fluid bed coating is the technology of choice for handling a tacky material, i.e., amorphous compound that cannot be processed by conventional aqueous processing technology. The amorphous compound is solubilized in ethanol and is converted into a stable amorphous form after removal of the ethanol.

The ratio of the therapeutically effective compound to the ionic water-insoluble polymer in general is from about 5:1 to about 1:5, preferably from about 4:1 to about 1:4, more preferably from about 3.5:1 to about 1:3.5, and most preferably from about 3:1 to about 1:3, respectively.

The therapeutically effective compound is present in the pharmaceutical solid dosage form in general in an amount of from about 5% to about 75%, preferably from about 10% to about 60%, more preferably from about 25% to about 50%, and most preferably from about 20% to about 40%, by weight of the total composition.

The therapeutically effective amount of the therapeutically effective compound is present in the pharmaceutical solid dosage form in an amount of from about 5 mg to about 750 mg, preferably from about 20 mg to about 500 mg, more preferably from about 50 mg to about 300 mg, and most preferably from about 100 mg to about 200 mg.

Preferably, the pharmaceutical solid dosage form is deposited on a microcrystalline cellulose sphere and further comprises a seal coat around the pharmaceutical solid dosage.

The ionic water-insoluble polymer matrix in general has a mean particle size of from about 100 microns to about 1500 microns, preferably from about 150 microns to about 1450 microns, more preferably from about 175 microns to about 1400 microns, and most preferably from about 200 microns to about 1375 microns.

In another preferred embodiment, the present invention provides a method for treating a disease comprising administering to a subject, in need thereof, a solid pharmaceutical dosage form for oral administration comprising a therapeutically effective amount of an unstable crystalline form or an amorphous form of a therapeutically effective compound micro-embedded into an ionic water-insoluble polymer, wherein the ratio of the therapeutically effective compound to the ionic water-insoluble polymer is from about 5:1 to about 1:5, respectively.

In yet another preferred embodiment, the present invention provides a method for preparing a pharmaceutical solid dosage form for oral administration which comprises micro-embedding an unstable crystalline form or an amorphous form of a therapeutically effective compound into an ionic water-insoluble polymer, wherein the ratio of the amorphous compound to the ionic polymer carrier is from about 5:1 to about 1:5, respectively.

The pharmaceutical solid dosage form of the present invention is prepared by a process, which preferentially converts the crystalline form of a therapeutically active compound into the amorphous form micro-embedded into an ionic water-insoluble polymer matrix. Preferably, the resulting granulation (i.e., beadlet) is blended or seal coated with an anti-tacking agent. The percentage of anti-tacking agent added to the spheres is from about 1% to about 5%.

The pharmaceutical dosage forms of the present invention can be prepared according to the examples set out below. The examples are presented for purposes of demonstrating, but not limiting, the preparation of the dosage forms of this invention.

### EXAMPLES

The following examples are provided to illustrate pharmaceutical solid dosage forms, which utilize (i) different ratios of amorphous compounds to ionic water-insoluble polymer; (ii) different types of the polymers (i.e., ionic water-insoluble polymers versus nonionic water-soluble polymers); and (iii) different physically unstable crystalline forms used as a starting material.

### Example 1

In this example, the inventive pharmaceutical solid dosage form of amorphous 2(R)-(3-chloro-4-methanesulfonyl-phenyl)-3-[1(R)-3-oxo-cyclopentyl]-N-(pyrazin-2-yl)-propionamide (Compound A) was prepared, wherein the amorphous drug was micro-embedded into an ionic water-insoluble polymer. Compound A IPA is the isopropyl alcohol solvate, which is a physically unstable crystalline form used as a starting material, and is converted to the amorphous form by the micro-embedding process.

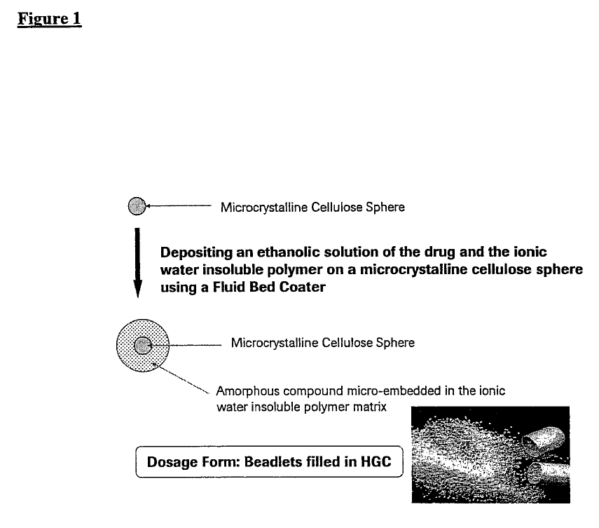

FIG. 1 is a diagram illustrating a preferred micro-embedding process for depositing an ethanolic solution of a therapeutically effective compound and an ionic water-insoluble polymer on a microcrystalline cellulose sphere using a fluid bed coater.

The excipients used in the formulation examples are set out below: Eudragit® L100 and Eudragit® L100-55 (Vendor—Rohm Pharma—Degussa).

Kollidon VA 64 (Vendor—BASF) Vinylpyrrolidone-vinyl acetate copolymer, Copolyvidone, copovidone, VPNAc copolymer 60/40, copolymer of 1-vinyl-2-pyrrolidone and vinyl acetate in a ratio of 6:4 by mass.

Amorphous Calcium Silicate (Zeopharm 600)—Vendor: Mutchler.

Cellets® (Vendor: Glatt Air Techniques) are Cellulose microcrystalline spheres prepared by pelletization.

Particle Size Specifications:

Cellets® 200: Particle Size: 200 to 355 μm: ≧85%.

Cellets® 350: Particle Size 350 to 500 μm: ≧85%.

Altalc-500 (Vendor: Luzenac America) is talc, very fine powder grade.

Corn Starch (Vendor: National Starch).

Povidone K30 (Vendor: BASF).

Formulation Composition

| | |
|---|---|
| Ingredients | mg per capsule* |

Drug Layering:

| | |
|---|---|
| Compound A IPA | 114.245** |
| Eudragit ® L100-55 | 66.67 |
| Cornstarch | 18.50 |
| Microcrystalline Cellulose Spheres | 256.33 |
| (Cellets-200) | |

Seal Coat:

| | |
|---|---|
| Amorphous Calcium Silicate | 8.55 |
| (Zeopharm 600) | |
| Povidone K30 | 0.45 |
| Fill Weight* | 450.50 |

Filled in hard gelatin capsule
**Equivalent to 100 mg anhydrous form after the IPA removal during processing

Drug Micro-Embedding Procedure
Preparation of the Drug Layering Suspension

In a tarred stainless steel container, add Compound A IPA to ethyl alcohol 200 proof while mixing using a propeller mixer at medium speed. Continue to mix until the Compound A IPA is completely dissolved. Slowly add the polymer to the above solution while mixing at medium speed. Continue to mix until the polymer is completely dissolved. Add cornstarch (or Altalc-500 as specified in the formulation) to the above solution while mixing using a propeller mixer at medium speed. Continue mixing for at least 1 hour or until a uniform dispersion of the drug layering suspension is obtained.

Application of the Drug Layering Suspension to Spheres

Place microcrystalline cellulose spheres (Cellets 200) into a fluid bed coater with a Wurster HS insert. Warm the microcrystalline cellulose spheres (for at least 2 minutes with inlet air temperature of 50°±15° C., providing sufficient air volume to fluidize the spheres. Spray the drug layering suspension from above onto the microcrystalline cellulose spheres mixing continuously using a propeller mixer at medium speed employing the following processing conditions:

| | |
|---|---|
| Inlet temperature | 50° ± 15° C. |
| Target product temperature | 40° ± 10° C. |
| Nozzle orifice | 1.0 ± 0.5 mm |
| Atomization air pressure | 3.0 ± 1.0 Bar |

Use sufficient air volume used to fluidize the spheres

Dry the resulting drug layered spheres for at least 1 hour prior to applying the seal coating process.

Seal Coating Procedure

Preparation of the Seal Coating Suspension

In a stainless steel container, add povidone K30 (polyvinyl pyrrolidone) to ethyl alcohol 200 proof while mixing using a propeller mixer at medium speed. Continue to mix until the povidone K30 is completely dissolved. Add amorphous calcium silicate (Zeopharm 600) to the above solution while mixing using a propeller mixer at medium speed for at least 30 minutes or until a uniform dispersion of the seal coating suspension is obtained.

Application of the Seal Coating Suspension to the Drug Layered Spheres

Spray the seal coating suspension from above mixing continuously using a propeller mixer at medium speed to the drug layered spheres from above using the following processing conditions:

| | |
|---|---|
| Inlet air temperature | 50° ± 15° C. |
| Target product temperature | 40° ± 10° C. |
| Nozzle orifice | 1.0 ± 0.5 mm |
| Atomization air pressure | 3.0 ± 1.0 Bar |

Use sufficient air volume used to fluidize the spheres.

Dry the seal coated spheres from above using an inlet air temperature of 40°±15° C. for at least 30 minutes. Cool the seal coated spheres to obtain a product temperature of 30°±5° C. by turning off the process air heat. Discharge the seal coated spheres into double polyethylene bags in an opaque high-density polyethylene pail. Ship the finished seal coated spheres in double polyethylene bags in a closed opaque high-density polyethylene pail with two silica gel bags between the polyethylene bags for encapsulation.

Encapsulation

Using a capsule-filling machine, fill the seal coated spheres from above into white opaque hard gelatin capsules at the specified target weight. Dedust the white opaque hard gelatin capsules as necessary. Store the finished white opaque hard gelatin capsules in double polyethylene bags in a closed opaque high-density polyethylene pail with two silica gel bags between the polyethylene bags.

### Example 2

In this example, a pharmaceutical solid dosage form of amorphous 2(R)-(3-chloro-4-methanesulfonyl-phenyl)-3-[1(R)-3-oxo-cyclopentyl]-N-(pyrazin-2-yl)-propionamide (Compound A) was prepared, wherein the amorphous compound was micro-embedded into a nonionic water-soluble polymer. Compound A IPA is the isopropyl alcohol solvate, which is a physically unstable crystalline form used as a starting material, and is converted to the amorphous form by the micro-embedding process.

Formulation Composition

| | |
|---|---|
| Ingredients | mg/capsule* |

Drug Layering:

| | |
|---|---|
| Compound A IPA | 114.245** |
| Kolidon ® VA 64 | 60.00 |
| Altalc-500 | 40.00 |
| Microcrystalline Cellulose Spheres | 117.46 |
| (Cellets-200) | |

Seal Coat:

| | |
|---|---|
| Amorphous Calcium Silicate | 6.40 |
| (Zeopharm 600) | |
| Fill weight* | 323.86 |

Filled in hard gelatin capsule
**Equivalent to 100 mg anhydrous form after the IPA removal during processing

Method of Preparation

The capsule was prepared in a manner similar to that set out in Example 1, except that Altalc-500, instead of cornstarch, was used as the anti-tacking agent. The seal coating procedure was replaced with the blending procedure by blending the resulting drug layered spheres with amorphous calcium silicate (Zeopharm 600) in a Turbula mixer for 5 minutes.

### Example 3

In this example, the inventive amorphous 2(R)-(3-chloro-4-methanesulfonyl-phenyl)-3-[1(R)-3-oxo-cyclopentyl]-N-(pyrazin-2-yl)-propionamide (Compound A) formulation was prepared with increased drug loading, wherein the amorphous drug was micro-embedded into an ionic water-insoluble polymer. Compound A IPA is the isopropyl alcohol solvate, which is a physically unstable crystalline form used as a starting material, and is converted to the amorphous form by the micro-embedding process.

| | |
|---|---|
| Ingredient | mg per capsule* |

Drug Layering:

| | |
|---|---|
| Compound A IPA | 114.245** |
| Eudragit ® L100-55 | 66.670 |
| Cornstarch | 18.500 |
| Microcrystalline Cellulose Spheres | 126.150 |
| (Cellets-200) | |

Seal Coat:

| | |
|---|---|
| Amorphous Calcium Silicate | 5.730 |
| (Zeopharm 600) | |
| PVP K30 | 0.620 |
| Fill weight* | 317.670 |

Filled in hard gelatin capsule
**Equivalent to 100 mg anhydrous form after the IPA removal during processing

The capsule was prepared in a manner similar to that set out in Example 1.

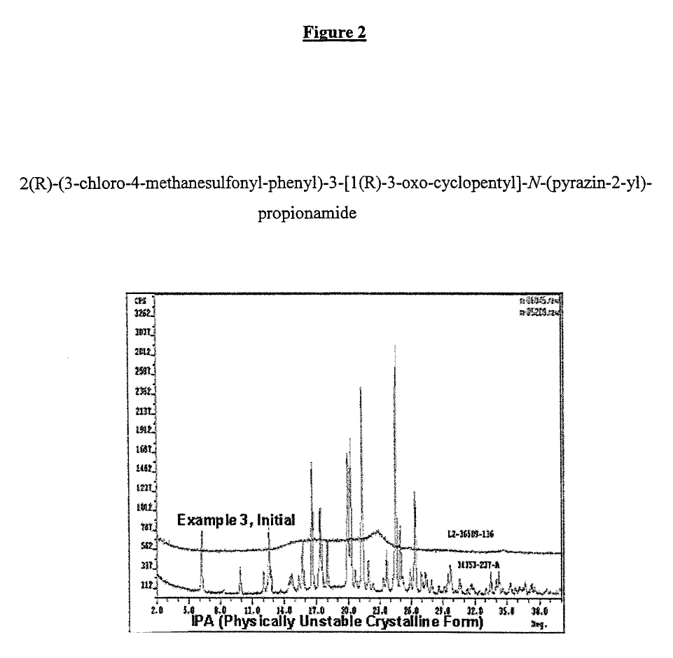

FIG. 2 is a graph illustrating the powder X-Ray pattern of the pharmaceutical solid dosage form of amorphous 2(R)-(3-chloro-4-methanesulfonyl-phenyl)-3-[1(R)-3-oxo-cyclopentyl]-N-(pyrazin-2-yl)-propionamide (Compound A) (Example 3) compared to the Compound A isopropanol solvate, a physically unstable crystalline form used as a starting material, indicating that the selected micro-embedding process preferentially converted the crystalline form to amorphous form.

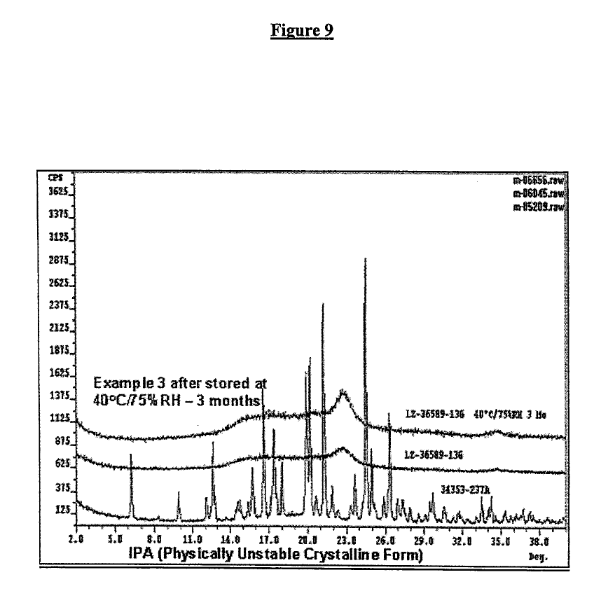

FIG. 9 is a graph illustrating the powder X-Ray patterns of the pharmaceutical solid dosage form of amorphous 2(R)-(3-chloro-4-methanesulfonyl-phenyl)-3-[1(R)-3-oxo-cyclopentyl]-N-(pyrazin-2-yl)-propionamide (Compound A) (Example 3) after 3-months storage at accelerated conditions (40° C./75% RH) in an induction-sealed opaque high density polyethylene bottle with a plastic cap, indicating that the compound remained in an amorphous form.

### Examples 4-7

In these examples, solid dosage forms of amorphous 2(R)-(3-chloro-4-methanesulfonyl-phenyl)-3-[1(R)-3-oxo-cyclopentyl]-N-(pyrazin-2-yl)-propionamide (Compound A), wherein the amorphous compound was micro-embedded either into ionic water-insoluble polymers or into nonionic water-soluble polymers in Examples 4-5 or Examples 6-7, respectively. These compositions were prepared to illustrate the effect of polymers on dissolution profiles of the dosage forms. Compound A IPA is the isopropyl alcohol solvate, which is a physically unstable crystalline form used as a starting material, and is converted to the amorphous form by the micro-embedding process.

Formulation Composition

| mg per capsule* | | | |
|---|---|---|---|
| Example 4 | Example 5 | Example 6 | Example 7 |

| | | |
|---|---|---|
| | Ionic-water-insoluble | Nonionic water-soluble |
| Ingredient | polymer | polymer |

Drug Layering:

| | | | | |
|---|---|---|---|---|
| Compound A | 114.245** | 114.245** | 114.245** | 114.245** |
| IPA | | | | |
| Eudragit ® L100- | 66.670 | — | — | — |
| 55 | | | | |
| Eudragit ® L100 | — | 66.670 | — | — |
| Povidone K30 | — | — | 66.670 | — |
| Klucel LF | — | — | — | 66.670 |
| Altalc-500 | 29.412 | 29.412 | 29.412 | 29.412 |
| Microcrystalline | 303.918 | 303.918 | 303.918 | 303.918 |
| Cellulose | | | | |
| Spheres | | | | |
| (Cellets-200) | | | | |

Seal Coat:

| | | | | |
|---|---|---|---|---|
| Amorphous | 10.204 | 10.204 | 10.204 | 10.204 |
| Calcium Silicate | | | | |
| (Zeopharm 600) | | | | |
| Fill weight* | 510.204 | 510.204 | 510.204 | 510.204 |

Filled in hard gelatin capsule
**Equivalent to 100 mg anhydrous form after IPA removal during processing

The capsule was prepared in a manner similar to that set out in Example 1, except that Altalc-500, instead of cornstarch, was used as anti-tacking agent. The seal coating procedure was replaced with the blending procedure by blending the resulting drug layered spheres with amorphous calcium silicate (Zeopharm 600) in a Turbula mixer for 5 minutes.

### Example 8

In this example, the inventive amorphous 2(R)-(3-chloro-4-methanesulfonyl-phenyl)-3-cyclopentyl-N-[5-(1(S),2-dihydroxyethyl)-pyrazin-2-yl]-propionamide (Compound B) formulation was prepared, wherein the amorphous drug was micro-embedded into an ionic water-insoluble polymer. Compound B is a physically unstable crystalline form used as a starting material and is converted to an amorphous form by the micro-embedding process.

Formulation Composition

| | |
|---|---|
| Ingredients | mg per capsule* |

Drug Layering:

| | |
|---|---|
| Compound B | 100.00 |
| Eudragit ® L100-55 | 66.67 |
| Cornstarch | 18.50 |
| Microcrystalline Cellulose Spheres | 67.18 |
| (Cellets-200) | |

Seal Coat:

| | |
|---|---|
| Amorphous Calcium Silicate | 4.65 |
| (Zeopharm 600) | |
| Povidone K30 | 0.50 |
| Fill Weight* | 257.50 |

Filled in Hard Gelatin Capsule

The capsule was prepared in a manner similar to that set out in Example 1.

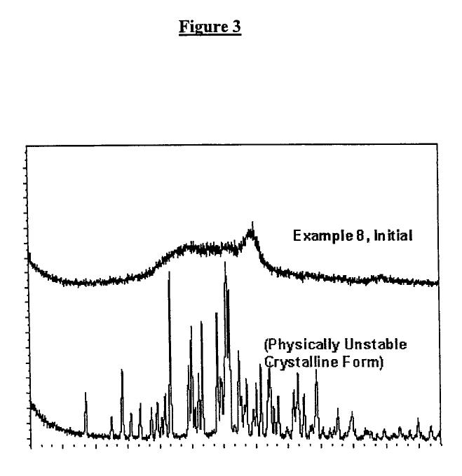

FIG. 3 is a graph illustrating the powder X-Ray patterns of the pharmaceutical solid dosage form of amorphous 2(R)-(3-chloro-4-methanesulfonyl-phenyl)-3-cyclopentyl-N-[5-(1(S),2-dihydroxyethyl)-pyrazin-2-yl]-propionamide (Compound B) (Example 8) compared to the physically unstable crystalline form of Compound B used as a starting material, indicating that the selected micro-embedding process preferentially converted the crystalline form to amorphous form.

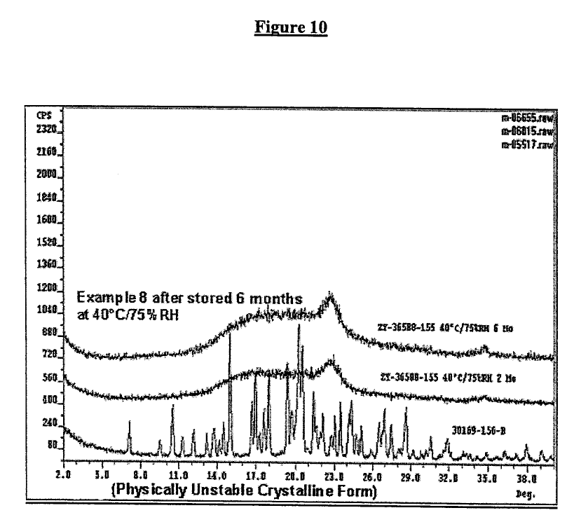

FIG. 10 is a graph illustrating the powder X-Ray patterns of a pharmaceutical solid dosage form of amorphous 2(R)-(3-chloro-4-methanesulfonyl-phenyl)-3-cyclopentyl-N-[5-(1(S),2-dihydroxyethyl)-pyrazin-2-yl]-propionamide (Compound B) (Example 8) after 6-month storage at accelerated conditions (40° C./75% RH) in an induction-sealed opaque high density polyethylene bottle with a plastic cap, indicating that the compound remained in an amorphous form.

### Example 9

In this example, an amorphous 2(R)-(3-chloro-4-methanesulfonyl-phenyl) -3-cyclopentyl-N-[5-(1(S),2-dihydroxyethyl)-pyrazin-2-yl]-propionamide (Compound B) formulation was prepared, wherein the amorphous drug was micro-embedded in a nonionic water-soluble polymer. Compound B is a physically unstable crystalline form used as a starting material and is converted to an amorphous form by the micro-embedding process.

Formulation Composition

| | |
|---|---|
| Ingredients | mg per capsule* |

Drug Layering:

| | |
|---|---|
| Compound B | 100.00 |
| Kollidon ® VA 64 | 50.00 |
| Cornstarch | 16.67 |
| Microcrystalline Cellulose Spheres | 297.58 |
| (Cellets-200) | |

Seal Coat:

| | |
|---|---|
| Amorphous Calcium Silicate | 3.00 |
| (Zeopharm 600) | |
| Fill Weight* | 467.25 |

Filled in hard gelatin capsule

The capsule was prepared in a manner similar to that set out in Example 1, except that the seal coating procedure was replaced with the blending procedure by blending the resulting spheres with amorphous calcium silicate (Zeopharm 600) in a Turbula mixer for 5 minutes.

### Examples 10-11

### Control Samples

In these examples, amorphous 2(R)-(3-chloro-4-methanesulfonyl-phenyl)-3-[1(R)-3-oxo-cyclopentyl]-N-(pyrazin-2-yl)-propionamide (Compound A) was prepared in a conventional manner. Compound A was physically mixed with either ionic water-insoluble polymer (i.e. Eudragit® L100-55, Eudragit® L100) or nonionic water-soluble polymer (i.e. Povidone K30, Klucel LF). Compound A was not micro-embedded into these polymers.

Formulation Composition

| | Example 10 | Example 11 |
|---|---|---|
| Ingredient | mg per capsule* | mg per capsule* |
| Compound A, spray-dried | 100.00 | 100.00 |
| powder | | |
| Eudragit L100-55 | 66.67 | — |
| Eudragit L100 | — | 66.67 |
| Povidone K30 | — | — |
| Klucel LF | — | — |
| Altalc-500 | 29.412 | 29.412 |
| Amorphous Calcium Silicate | 3.398 | 3.398 |
| (Zeopharm 600) | | |
| FILL WEIGHT* | 199.48 | 199.48 |

Filled in hard gelatin capsule

The capsule was prepared by weighing the spray dried Compound A powder, polymer, talc, and Zeopharm 600 and placing them in a blender. The mixture was blended for 10 minutes. The powder mix was screened through a sieve # 30 mesh and remixed in the blender for 5 minutes. A quantity of 199.48 mg of the powder mix was filled into a hard gelatin capsule size #0.

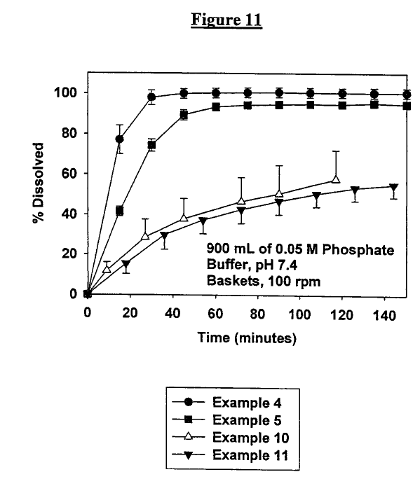

FIG. 11 is a graph illustrating a comparison between the dissolution profiles of the inventive pharmaceutical solid dosage form of Compound A prepared by the micro-embedding process using ionic water-insoluble polymer in Examples 4-5 and the solid dosage form of Compound A prepared in Examples 10-11 by a conventional process (physical mix; non-micro-embedding process).

This Example illustrates that the micro-embedding process of the unstable crystalline form of the compound into the ionic water-insoluble polymer provides a relatively fast, complete dissolution profiles. In contrast, the conventional formulation (physical mix; non-micro-embedding process) provided an inferior dissolution profile.

### Example 12

### Control Sample

In this example, unstable crystalline 2(R)-(3-chloro-4-methanesulfonyl-phenyl)-3-cyclopentyl-N-[5-(1(S),2-dihydroxyethyl)-pyrazin-2-yl]-propionamide (Compound B) was prepared in a conventional manner. Compound B was physically mixed with Eudragit® L100-55. Compound B was not micro-embedded into the ionic water-insoluble polymer.

Formulation Composition

| Ingredients | mg per capsule* |
|---|---|
| Compound B, micronized powder | 100.00 |
| Eudragit L100-55 | 66.67 |
| Cornstarch | 18.50 |
| Amorphous Calcium Silicate | 4.65 |
| (Zeopharm 600) | |
| Povidone K30 | 0.50 |
| FILL WEIGHT* | 190.32 |

Filled in hard gelatin capsule

The capsule was prepared by weighing the micronized Compound B powder, Eudragit L-100-55 and cornstarch and placing them in a blender. The mixture was blended for 5 minutes. Zeopharm 600 and PVP K30 were then added to the blender and the mixture further blended for 2 minutes. A quantity of 190.32 mg of the powder mix was filled into a hard gelatin capsule size #0.

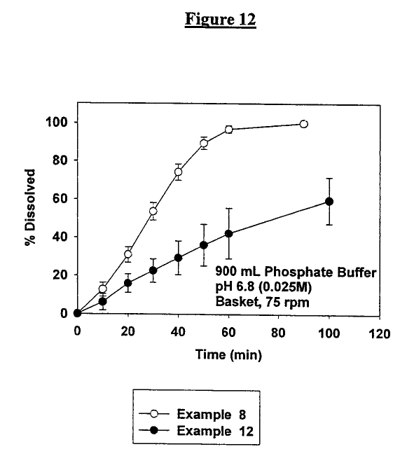

FIG. 12 is a graph illustrating a comparison between the dissolution profiles of the inventive pharmaceutical solid dosage form of Compound B prepared by the micro-embedding process using ionic water-insoluble polymer in Example 8 and the solid dosage form of Compound B prepared in Example 12 by a conventional process (physical mix; non-micro-embedding process).

FIGS. 11-12 illustrate that the micro-embedding process of the unstable crystalline form or amorphous form of the compound into the ionic water-insoluble polymer provides a relatively fast, complete dissolution profiles. In contrast, the conventional formulation (physical mix; non-micro-embedding process) provided an inferior dissolution profile.

Dissolution Testing

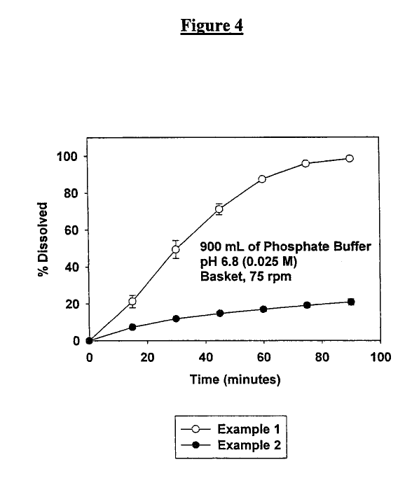

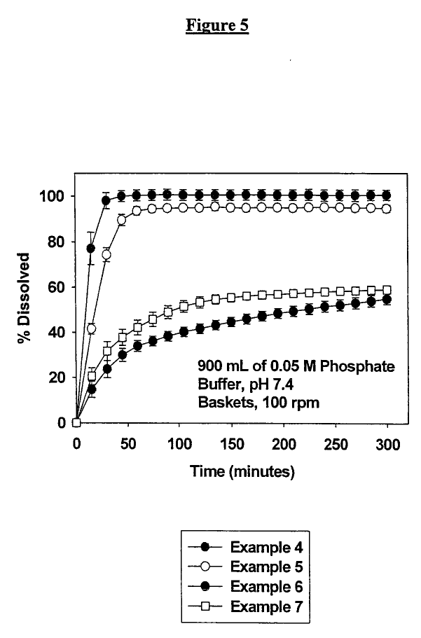

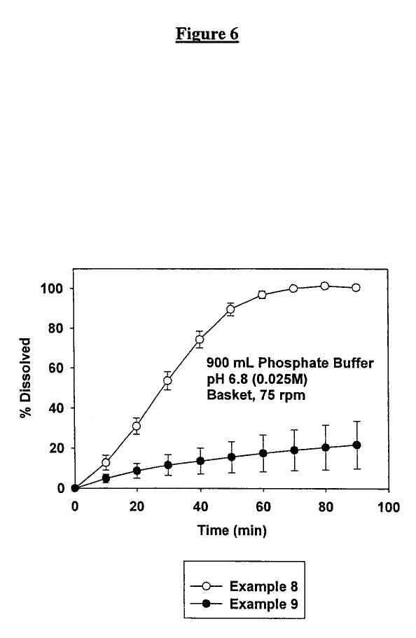

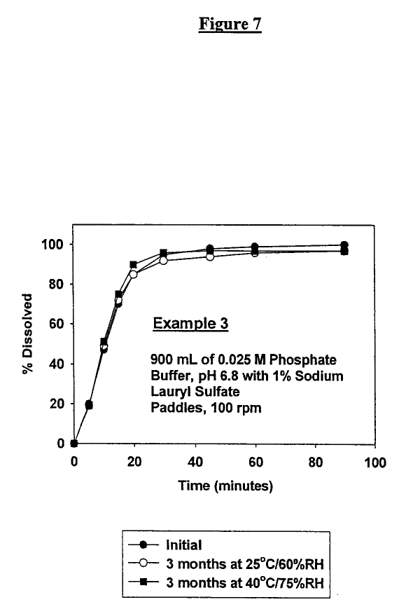

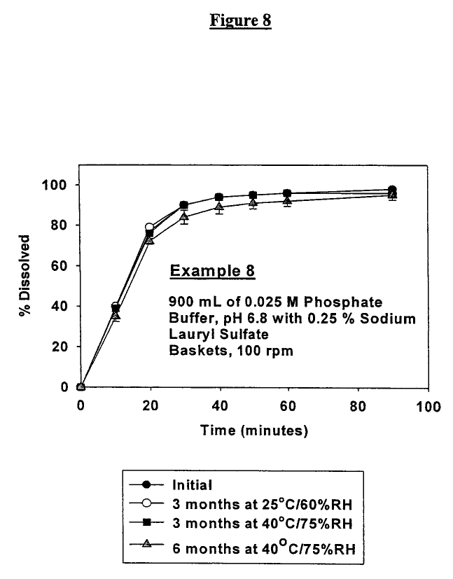

Oral dosage forms containing Compound A (Examples 1-7 and 10-11) and Compound B (Examples 8-9 and 12) were evaluated for dissolution in 900 mL of a dissolution medium using a USP apparatus (basket or paddle method) at specified speeds. Sample aliquots were taken at different time intervals and analyzed by UV or HPLC. The results of the dissolution studies and the medium, method, and speeds are set out in FIGS. 4-8.

The inventive formulations, in which an amorphous drug (Compound A or Compound B) was micro-embedded in the ionic water-insoluble polymer, provided relatively fast, complete dissolution profiles (Examples 1, 3, 4, 5, and 8). The ionic water-insoluble polymer does protect the amorphous drug from gelling when exposed to dissolution media. In contrast, the conventional formulations, in which an amorphous drug (Compound A or Compound B) was micro-embedded into the non-ionic water-soluble polymer, provided relatively slow, incomplete dissolution profiles (Examples 2, 6, 7, and 9). This data shows that the non-ionic-water soluble polymer does not protect the amorphous drug from gelling when exposed to dissolution media. The inventive pharmaceutical solid dosage forms protect the amorphous drug from the microenvironments, thereby maintaining dissolution characteristics of the dosage form even under the stressed storage conditions (i.e., 3-6 months at 40° C./75% RH).

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

What is claimed is:

1. A pharmaceutical solid dosage form for oral administration comprising a therapeutically effective amount of a physically unstable crystalline form or an amorphous form of a therapeutically effective compound micro-embedded into an ionic water-insoluble polymer, wherein the ratio of the therapeutically effective compound to the ionic water-insoluble polymer is from about 5:1 to about 1:5, respectively.

2. The dosage form according to claim 1, wherein the therapeutically effective compound is a glucokinase activator compound.

3. The dosage form according to claim 2, wherein the glucokinase activator compound is 2(R)-(3-chloro-4-methanesulfonyl-phenyl)-3-[1(R)-3-oxo-cyclopentyl]-N-(pyrazin-2-yl)-propionamide or 2(R)-(3-chloro-4-methanesulfonyl-phenyl)-3-cyclopentyl-N-[5-(1(S),2-dihydroxyethyl)-pyrazin-2-yl]-propionamide.

4. The dosage form according to claim 1, wherein the therapeutically effective compound is present in the pharmaceutical solid dosage form in an amount of from about 5% to about 75%, by weight of the total composition.

5. The dosage form according to claim 1, wherein the therapeutically effective amount of the therapeutically effective compound is present in the pharmaceutical solid dosage form in an amount of from about 5 mg to about 750 mg.

6. The dosage form according to claim 1, wherein the ionic water-insoluble polymer has a molecular weight ranging from about 60,000 to about 300,000 Daltons.

7. The dosage form according to claim 6, wherein the ionic water-insoluble polymer is selected from the group consisting of methacrylic acid and ethyl acrylate copolymers, methacrylic acid and methylmethacrylate copolymers, dimethylaminoethylmethacrylate and neutral methacrylic ester copolymers, cellulose acetate phthalates, polyvinyl acetate phthalates, hydroxylpropyl methylcellulose phthalates, and hydroxylpropyl methylcellulose acetate succinates.

8. The dosage form according to claim 7, wherein the ionic water-insoluble polymer is a methacrylic acid and methylmethacrylate copolymer or a methacrylic acid and ethyl acrylate copolymer.

9. The dosage form according to claim 1, wherein the pharmaceutical solid dosage form is deposited on a microcrystalline cellulose sphere.

10. The dosage form according to claim 1, further comprising a seal coat around the pharmaceutical solid dosage.

11. A method for treating a disease comprising administering to a subject, in need thereof, a solid pharmaceutical dosage form for oral administration comprising a therapeutically effective amount of physically unstable crystalline form or an amorphous form of a therapeutically effective compound micro-embedded into an ionic water-insoluble polymer, wherein the ratio of the therapeutically effective compound to the ionic water-insoluble polymer is from about 5:1 to about 1:5, respectively.

12. The method according to claim 11, wherein the therapeutically effective compound is a glucokinase activator compound.

13. The method according to claim 12, wherein the glucokinase activator compound is 2(R)-(3-chloro-4-methanesulfonyl-phenyl)-3-[1(R)-3-oxo-cyclopentyl]-N-(pyrazin-2-yl)-propionamide or 2(R)-(3-chloro-4-methanesulfonyl-phenyl)-3-cyclopentyl-N-[5-(1(S),2-dihydroxyethyl)-pyrazin-2-yl]-propionamide.

14. The method according to claim 11, wherein the therapeutically effective compound is present in the pharmaceutical solid dosage form in an amount of from about 5% to about 50%, by weight of the total composition.

15. The method according to claim 11, wherein the therapeutically effective amount of the therapeutically effective compound is present in the pharmaceutical solid dosage form in an amount of from about 5 mg to about 750 mg.

16. The method according to claim 11, wherein the ionic water-insoluble polymer is selected from the group consisting of methacrylic acid and ethyl acrylate copolymers, methacrylic acid and methylmethacrylate copolymers, dimethylaminoethylmethacrylate and neutral methacrylic ester copolymers, cellulose acetate phthalates, polyvinyl acetate phthalates, hydroxylpropyl methyl cellulose phthalates, and hydroxylpropyl methyl cellulose acetate succinates.

17. A method for preparing a pharmaceutical solid dosage form for oral administration which comprises micro-embedding a therapeutically effective amount of an unstable crystalline form or an amorphous form of a therapeutically effective compound into an ionic water-insoluble polymer, wherein the ratio of the amorphous compound to the ionic polymer carrier is from about 5:1 to about 1:5, respectively.

18. The method according to claim 17, wherein the therapeutically effective compound is a glucokinase activator compound.

19. The method according to claim 18, wherein the glucokinase activator compound is 2(R)-(3-chloro-4-methanesulfonyl-phenyl)-3-[1(R)-3-oxo-cyclopentyl]-N-(pyrazin-2-yl)-propionamide or 2(R)-(3-chloro-4-methanesulfonyl-phenyl)-3-cyclopentyl-N-[5-(1(S),2-dihydroxyethyl)-pyrazin-2-yl]-propionamide.

20. The method according to claim 17, wherein the therapeutically effective compound is present in the pharmaceutical solid dosage form in an amount of from about 5% to about 50%, by weight of the total composition.

21. The method according to claim 17, wherein the therapeutically effective amount of the therapeutically effective compound is present in the pharmaceutical solid dosage form in an amount of from about 5 mg to about 750 mg.

22. The method according to claim 17, wherein the ionic water-insoluble polymer is selected from the group consisting of methacrylic acid and ethyl acrylate copolymers, methacrylic acid and methylmethacrylate copolymers, dimethylaminoethylmethacrylate and neutral methacrylic ester copolymers, cellulose acetate phthalates, polyvinyl acetate phthalates, hydroxylpropyl methyl cellulose phthalates, and hydroxylpropyl methyl cellulose acetate succinates.

23. The method according to claim 17, wherein the micro-embedding is selected from the group consisting of fluid bed coating, spray drying, lyophilizing, solvent-controlled microprecipitation, hot melt extrusion, and supercritical fluid evaporation.

24. The method according to claim 23, wherein the micro-embedding is fluid bed coating.

25. The method according to claim 17, wherein the micro-embedding converts a physically unstable crystalline form of a therapeutically active compound into an amorphous form.
